(19) United States
(12) Patent Application Publication
(10) Pub. No.: US 2024/0153601 A1
(43) Pub. Date: May 9, 2024

(54) TEXT DATA GENERATION METHOD

(71) Applicant: NEC Corporation, Minato-ku, Tokyo (JP)

(72) Inventors: Yuji OHNO, Tokyo (JP); Masahiro KUBO, Tokyo (JP)

(73) Assignee: NEC Corporation, Minato-ku, Tokyo (JP)

(21) Appl. No.: 18/278,970

(22) PCT Filed: May 20, 2021
(86) PCT No.: PCT/JP2021/019255

§ 371 Date: Aug. 25, 2023

(51) Int. Cl. G16H 10/60

(52) U.S. Cl. CPC G16H 10/60

(57) ABSTRACT

A text data generation device 100 includes an acquisition unit 121 that acquires object person information representing time-series physical condition of an object person, an estimation unit 122 that estimates time-series states of the object person on the basis of the object person information, and a generation unit 123 that generates text data representing the state of the object person in a given time period, on the basis of the estimated time-series states of the object person.

### TECHNICAL FIELD

The present invention relates to a text data generation method, a text data generation device, and a program.

### BACKGROUND ART

In a hospital, daily condition of a patient is recorded on an electronic medical record (hereinafter also referred to as “medial record”) in text data, in order that doctors and nurses grasp the state of the patient. For example, on a medical record, objective data representing the condition of a patient, obtained from physical examination or check-up of the patient, is recorded.

Patent Literature 1 discloses art of detecting a restless state that is an exemplary state of a patient. In Patent Literature 1, a restless state is detected from time-series data of biological information measured from a patient.

### CITATION LIST

### Patent Literature

Patent Literature 1: WO 2019/073927 A

### SUMMARY OF INVENTION

### Technical Problem

However, in the above-described art, even though a restless state of a patient is detected, a nurse or a doctor must record the state on a medical record or the like, which may cause a workload. Moreover, erroneous record (omission of record or incorrect record) of a state of a patient on a medical record or the like may be caused by a nurse or the like.

In view of the above, an object of the present invention it to provide a text data generation method capable of solving the problems described above.

### Solution to Problem

A text data generation method that is one aspect of the present invention is configured to include
acquiring object person information representing time-series physical condition of an object person;
estimating time-series states of the object person on the basis of the object person information; and
generating text data representing a state of the object person in a given time period on the basis of the estimated time-series states of the object person.

Further, a text data generation device that is one aspect of the present invention is configured to include
an acquisition unit that acquires object person information representing time-series physical condition of an object person;
an estimation unit that estimates time-series states of the object person on the basis of the object person information; and
a generation unit that generates text data representing a state of the object person in a given time period on the basis of the estimated time-series states of the object person.

Further, a program that is one aspect of the present invention is configured to cause an information processing device to execute processing to:
acquire object person information representing time-series physical condition of an object person;
estimate time-series states of the object person on the basis of the object person information; and
generate text data representing a state of the object person in a given time period on the basis of the estimated time-series states of the object person.

### Advantageous Effects of Invention

Since the present invention is configured as described above, it is possible to mitigate a load placed on the nurses so as to allow them to provide appropriate nursing.

### BRIEF DESCRIPTION OF DRAWINGS

FIG. 1 is a block diagram illustrating the overall configuration of an information processing system according to a first exemplary embodiment of the present invention.

FIG. 2 is a block diagram illustrating a configuration of a text data generation device constituting the text data generation system disclosed in FIG. 1.

FIG. 3 illustrates a state of processing by the text data generation device disclosed in FIG. 2.

FIG. 4 illustrates a state of processing by the text data generation device disclosed in FIG. 2.

FIG. 5 illustrates a state of processing by the text data generation device disclosed in FIG. 2.

FIG. 6 illustrates a state of processing by the text data generation device disclosed in FIG. 2.

FIG. 7 illustrates a state of processing by the text data generation device disclosed in FIG. 2.

FIG. 8 is a flowchart illustrating an operation of the text data generation device disclosed in FIG. 2.

FIG. 9 is a block diagram illustrating a hardware configuration of a text data generation device according to a second exemplary embodiment of the present invention.

FIG. 10 is a block diagram illustrating a configuration of the text data generation device according to the second exemplary embodiment of the present invention.

FIG. 11 is a flowchart illustrating an operation of the text data generation device according to the second exemplary embodiment of the present invention.

### DESCRIPTION OF EMBODIMENTS

### First Exemplary Embodiment

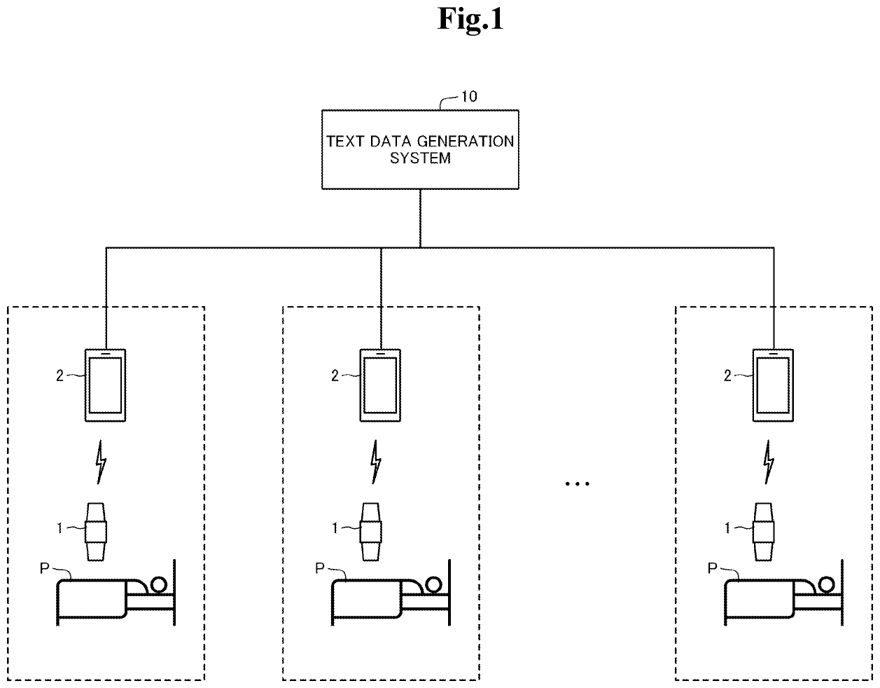

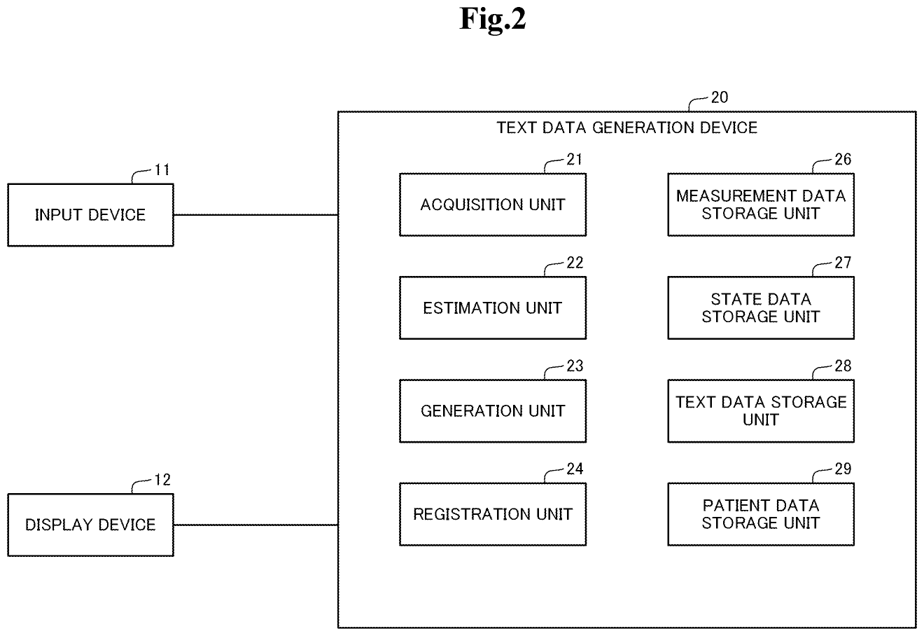

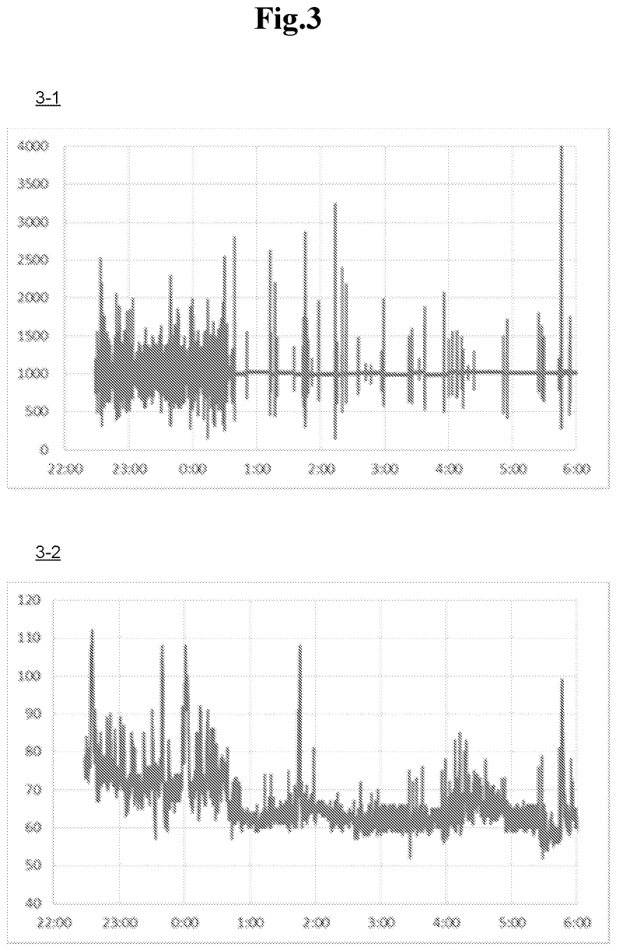

A first exemplary embodiment of the present invention will be described with reference to FIGS. 1 to 8. FIGS. 1 and 2 are diagrams for explaining a configuration of an information processing system, and FIGS. 3 to 8 are illustrations for explaining processing operation of the information processing system.

### [Configuration]

An information processing system of the present invention is used for obtaining time-series measurement data such as biological information from a patient P in a hospital, and generating, from the measurement data, text data representing the state of the patient in a given time period in text. The information processing system is configured to register generated text data with, for example, an electronic medical record of the patient, to thereby suppress errors in recording the state of the patient by medical professionals such as nurses and doctors taking care of the patient in the hospital.

While the case of using the information processing system of the present invention in a hospital will be described below as an example, the system may be used at nursing facilities or at home. In such a case, the information processing system is used to obtain measurement data such as biological information of a care-receiver at a nursing facility or home, and generate text data representing the state of the care-receiver. In that case, the generated text data is, for example, displayed and output to the staff of the nursing facility or to the family of the care-receiver, or transmitted to a system of the hospital managing the care-receiver, and displayed and output to the medical professionals and registered with the electronic medical record. Hereinafter, a detailed configuration of the information processing system will be described.

As illustrated in FIG. 1, an information processing system includes a sensor 1, a communication device 2, and a text data generation system 10. The sensor 1 is used to measure measurement data (object person information) representing the time-series physical condition of the patient P, from the patient P in a hospital. For example, the sensor 1 is a wearable device to be worn on a wrist of the patient P like a watch (for example, a smartwatch, an activity tracker, an activity meter), a bio sensor installed near the bed of the patient P (for example, a sheet-type sensor, a mat-type sensor, a radio-type sensor), a camera for capturing a video of the patient P, or the like. As time-series measurement data representing the physical condition of the patient P, the sensor 1 measures physical information that can be represented in numerical values such as heart rate, body temperature, blood pressure, blood oxygen saturation (SpO₂), respiration rate, acceleration (active mass), the number of steps, and the number of going up and down, and image information such as video images in which the behavior of the patient P is captured, and transmits the measurement data to the communication device 2 via wireless communication.

The communication device 2 is configured of, for example, a mobile communication terminal such as a wireless router or a smartphone. The communication device 2 receives measurement data of the patient P measured and transmitted by the sensor 1, and transfers it to the text data generation system 10.

As illustrated in FIG. 2, the text data generation system 10 includes an input device 11, a display device 12, and a text data generation device 20. The input device 11 is, for example, a keyboard and a mouse in which text input and operation on operation buttons can be made by a nurse or the like who is an operator. The display device 12 is, for example, a display, which displays and outputs text data generated by the text data generation device 20 as described below. The text data generation device 20 is configured of one or a plurality of information processing devices each having an arithmetic device and a storage device. As illustrated in FIG. 2, the text data generation device 20 includes an acquisition unit 21, an estimation unit 22, a generation unit 23, and a registration unit 24. The functions of the acquisition unit 21, the estimation unit 22, the generation unit 23, and the registration unit 24 can be implemented through execution, by the arithmetic device, of a program for implementing the respective functions stored in the storage device. The text data generation device 20 also includes a measurement data storage unit 26, a state data storage unit 27, a text data storage unit 28, and a patient data storage unit 29. The measurement data storage unit 26, the state data storage unit 27, the text data storage unit 28, and the patient data storage unit 29 are configured of storage devices. However, the measurement data storage unit 26, the state data storage unit 27, the text data storage unit 28, and the patient data storage unit 29 are not necessarily configured of storage devices of the text data generation device 20. They may be configured of storage devices outside the text data generation system 10. Hereinafter, the respective constituent elements will be described in detail.

The acquisition unit 21 acquires measurement data of the patient P measured by the sensor 1 as described above, via the communication device 2. Then, the acquisition unit 21 stores the acquired measurement data for each patient P, in the measurement data storage unit 26. For example, as measurement data measured for each patient P, the acquisition unit 21 acquires physical information that can be represented in numerical values such as heart rate, body temperature, blood pressure, blood oxygen saturation (SpO₂), respiration rate, acceleration (active mass), the number of steps, and the number of going up and down, and image information such as video images in which behavior of the patient P is captured. In particular, measurement data is time-series data including time information at which the measurement data is measured. For example, as illustrated in FIG. 3, measurement data is one in which acceleration (FIG. 3 (3-1)) and heart rate (FIG. 3 (3-2)) are shown on the vertical axis, with respect to the time shown in the horizontal axis.

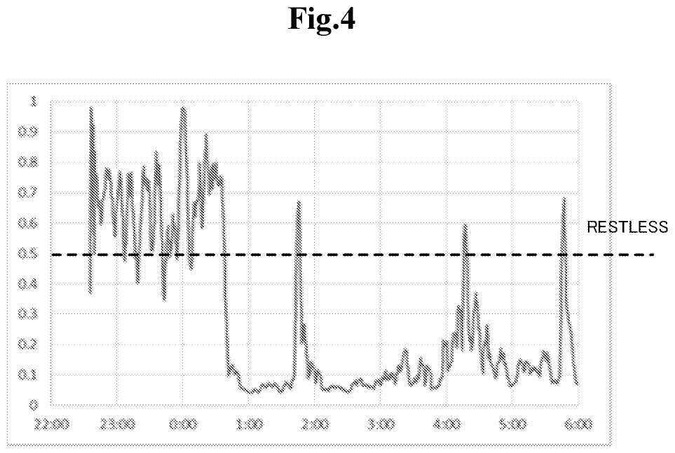

The estimation unit 22 estimates time-series states of the patient P from the measurement data of the patient P stored in the measurement data storage unit 26. Then, the estimation unit 22 stores the information representing the estimated states for each patient P, in the state data storage unit 27. For example, as time-series states of the patient P, the estimation unit 22 estimates a restless level, an awakening level, a sleeping level, an active mass level, whether the pulse is normal or abnormal, whether the cardiac function is normal or abnormal, whether the respiratory function is normal or abnormal, whether the cerebral function is normal or abnormal, a stress level, a numerical value of Glasgow Coma Scale (GCS), a numerical value of Japan Coma Scale (JCS), and the like. As an example, the estimation unit 22 calculates a score representing the degree that the patient is in a restless state on the basis of acceleration and heart rate that are measurement data corresponding to the time shown on the horizontal axis and shows the score on the vertical axis as illustrated in FIG. 4, and when the score is equal to or larger than a threshold (for example, 0.5), the estimation unit 22 estimates that the patient P is restless. As another example, the estimation unit 22 estimates whether the cardiac function is normal or abnormal on the basis of a time-series change in the blood pressure that is measurement data (for example, whether in an ascending direction of a descending direction).

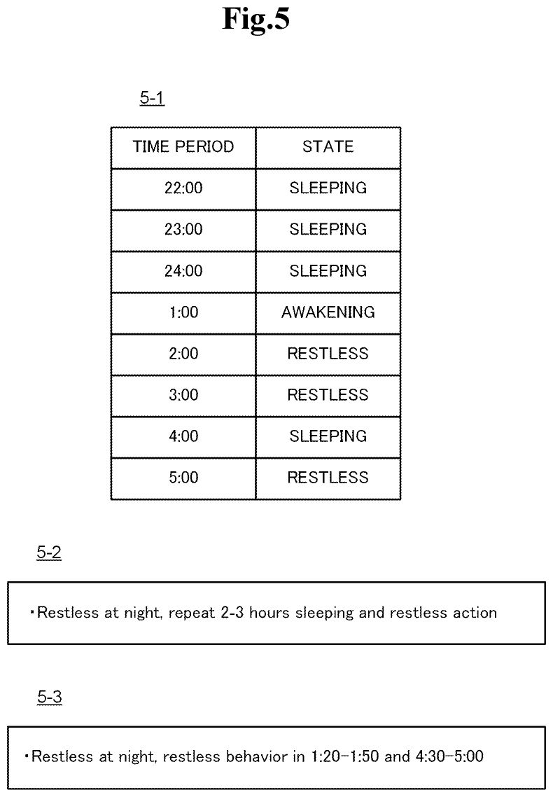

The generation unit 23 generates text data representing the state of the patient P in a given time period, on the basis of the estimated time-series states of the patient P stored in the state data storage unit 27. Then, the generation unit 23 stores the generated text data for each patient P, in the text data storage unit 28. For example, the estimation unit 22 generates text data representing the state of the patient P for each hour as illustrated in FIG. 5 (5-1) from the score data representing the time-series degree of restless state as illustrated in FIG. 4. That is, in the example of FIG. 5 (5-1), as the state of the patient P, the estimation unit 22 generates text data in which the state is “sleeping” in a time period “22:00”, “awakening” in a time period “1:00”, “restless” in a time period “2:00”, and the like. While the unit of “time period” is one hour in this example, the unit of “time period” may have any length of time such as several tens minutes or several hours.

Note that the generation unit 23 may generate text data representing the state of the patient P in a given time period, on the basis of a plurality of states among the estimated time-series states of the patient P. For example, from a plurality of estimated states of X minutes before, that is, immediately before the given time period, the generation unit 23 generates text data representing the state of the patient P in a given time period thereafter. As an example, the generation unit 23 generates text data representing the state of the patient P in accordance with a predetermined rule as described below, on the basis of a restless level, an awakening level, a sleeping level, and an active mass level that are a plurality of estimated time-series states of the patient P. For example, the generation unit 23 generates text data according to a rule that in X minutes before a given time period, when 80 percent is sleeping, the state is “sleeping”, when 80 percent is somnolence, the state is “somnolence”, when 50 percent or more is restless, the state is “restless”, when 50 percent or more is awakening and active, the state is “awakening”, when 50 to 80 percent is sleeping or somnolence and 50 to 20 percent is awakening, the state is “repeating sleeping and somnolence”, and associate the data with the given time period. When generating the text data, the generation unit 23 is not limited to use the estimated state of the patient P of the time before the given time period. The generation unit 23 may use an estimated state of the patient P at a predetermined time with respect to a given time period, such as an estimated state of the patient P during the given time period or an estimated state of the patient P after the given time period. As described above, the generation unit 23 may generate text data representing the state of the patient in a given time period, corresponding to the percentage of the state of the patient P estimated in a predetermined time with respect to the given time period.

Further, as illustrated in FIG. 5 (5-1), the generation unit 23 may also generate text data representing the state of the patient P in a time period combining a plurality of time periods on the basis of text data representing the states of the patient P in a plurality of time periods such as every hour. For example, like text data expressed in a sentence that “restless at night, repeat 2-3 hours sleeping and a restless state” as illustrated in FIG. 5 (5-2), the generation unit 23 may generate text data representing that states such as “restless” and “repeat sleeping and restless” have occurred in time periods of “night” and “2-3 hours”. However, the generation unit 23 is not limited to generate text data as illustrated in FIG. 5 (5-2) from text data representing the states of the patient Pin a plurality of time periods as illustrated in FIG. 5 (5-1), and may generate it from one or a plurality of states of the patient P estimated by the estimation unit 22. Moreover, the generation unit 23 may generate text data representing the state of the patient P in a shorter time period, on the basis of the estimated time-series state of the patient P as illustrated in FIG. 4 or time-series measurement data. For example, in text data in a sentence, that is, “restless behavior in 1:20-1:50 and 4:30-5:00” as illustrated in FIG. 5 (5-3), the generation unit 23 may generate text data representing the state of the patient P in a time period of minutes, rather than a time period of one hour.

Note that the generation unit 23 may generate text data representing a state of the patient P in a given time period by inputting the estimated state of the patient P or measurement data to a learning model generated in advance. In that case, a learning model is generated through learning by using, as training data, the state of the patient estimated as described above and the measurement data, and text data representing the state of the patient P recorded on an electronic medical record by a nurse who took care of the patient P. Note that text data stored on an electronic medical record may correspond to, for example, predetermined items in a nursing record, and objective data representing information obtained from physical examination or check-up that is 0 (objective) item in a program-oriented medical record called SOAP.

The registration unit 24 registers the text data stored in the text data storage unit 28 with an electronic medical record for each patient P stored in the patient data storage unit 29. In particular, the registration unit 24 registers the text data with the electronic medical record together with time data showing the given time period to which the text data representing the state of the patient corresponds. Note that the registration unit 24 is not limited to register text data with an electronic medical record, and may register with another data file set in advance.

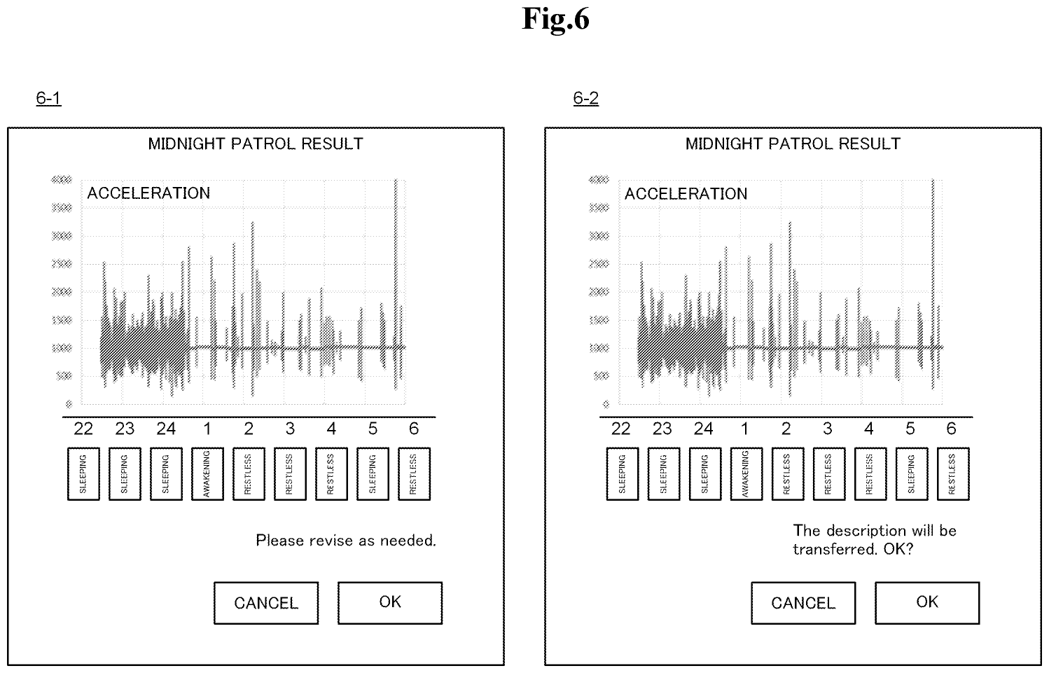

Specifically, before registering text data, the registration unit 24 first displays and outputs text data of the patient P stored in the text data storage unit 28 on the display device 12 so as to be viewable by a registered person who is a nurse having a relation with the patient P because the nurse takes care of the patient. As an example, as illustrated in FIG. 6 (6-1), the registration unit 24 displays and outputs text data representing a state (for example, sleepless, awakening, restless) of the patient P for each time period corresponding to the time set in the horizontal axis, in a text box set for each time period. At that time, the registration unit 24 displays the measurement data of the patient P stored in the measurement data storage unit 26 corresponding to the time in the horizontal axis, together with the text data. For example, in the example of FIG. 6 (6-1), measurement data of acceleration is displayed corresponding to the time shown in the horizontal axis. Moreover, the registration unit 24 outputs the text data shown in the text box so as to be revisable, and displays text “please revise as needed” to urge the nurse to revise it. Thereby, when the nurse revises the text data in the text box using the input device 11, the registration unit 24 accepts the input of the revised text data. Then, when the nurse performs a registration operation by using the input device 11, the registration unit 24 registers the text data input in the text box with the electronic medical record of the patient P together with the time data. For example, the registration unit 24 displays “The description will be transferred. OK?” on the screen as illustrated in FIG. 6 (6-2). Then, when the nurse operates the “OK” button, the registration unit 24 registers the text data input in the text box with the electronic medical record of the patient P together with the time data.

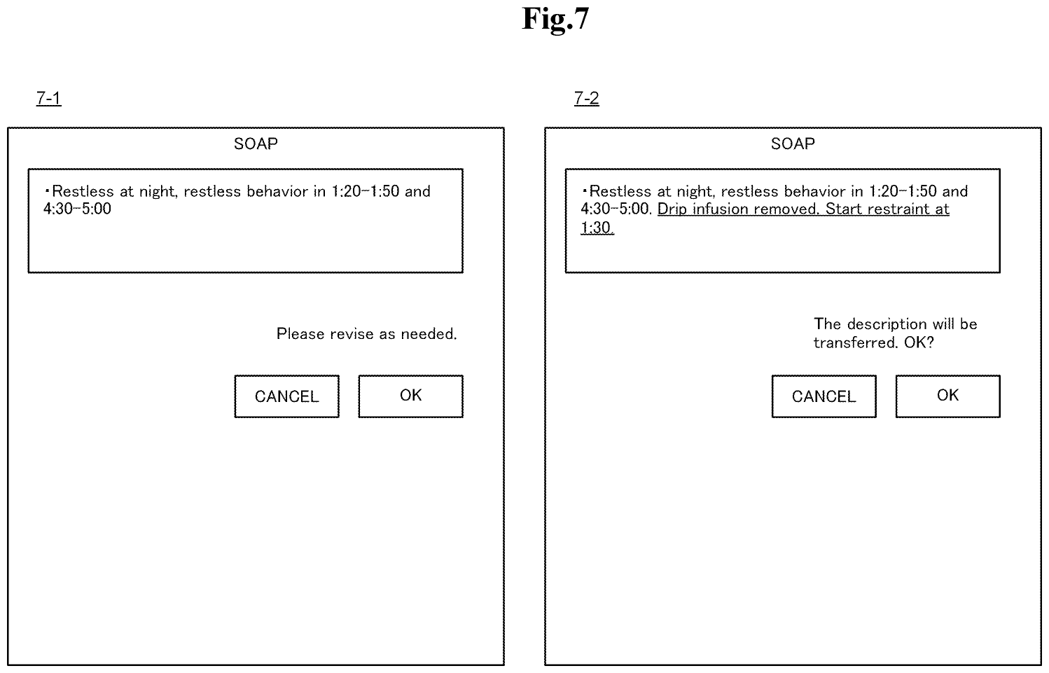

Here, another example of registration of text data by the registration unit 24 will be described with reference to FIG. 7. In the case where text data is shown in a sentence, the registration unit 24 outputs the text data in a sentence so as to be revisable in the text box as illustrated in FIG. 7 (7-1). The registration unit 24 further displays text “please revise as needed” to urge the nurse to revise. Accordingly, when the nurse revises the text data in the text box using the input device 11, the registration unit 24 accepts the input of the revised text data. For example, as shown with the underline in the text box of FIG. 7 (7-2), in the case where text data “Drip infusion removed. Start restraint at 1:30.” is added and then “OK” button is operated, the registration unit 24 registers the text data input in the text box in a predetermined field of the electronic medical record of the patient P. At that time, the registration unit 24 registers the text data expressed in a sentence as objective data representing the information obtained from physical examination or check-up that is 0 (objective) item of a problem-oriented medical record called SOAP in the electronic medical record. Note that the registration unit 24 may register the measurement data of the patient P in a predetermined field in the electronic medical record of the patient P together with information corresponding to the measured time. For example, the registration unit 24 may record the data like “2021/5/XX morning, heart rate 95 bpm” in the electronic medical record. Thereby, the measurement data can be recorded directly with the electronic medical record.

Note that when displaying the text data expressed in a sentence as illustrated in FIG. 7 on the display device 12, the registration unit 24 may display and output measurement data of the patient P together with the text data. For example, the registration unit 24 may display and output the measurement data in a form as illustrated in FIG. 6. The registration unit 24 may also display and output the text data representing the state of the patient P for each time period as illustrated in FIG. 6, the text data in a sentence as illustrated in FIG. 7, and the measurement data at the same time. Note that the registration unit 24 may display and output image information (for example, video images) in which the patient P is captured, together with the text data and the measurement data. Thereby, a nurse can revise and register the text data with reference to the measurement data.

### [Operation]

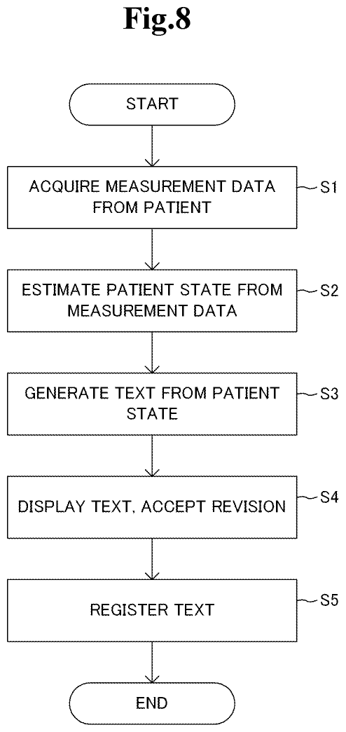

Next, operation of the text data generation device 20 described above will be described with reference to the flowchart of FIG. 8.

First, the text data generation device 20 acquires measurement data of the patient P measured by the sensor 1 attached to the patient P or installed near the bed or in the room of the patient P, via the communication device 2 (step S1). For example, as time-series measurement data of the patient P, the text data generation device 20 acquires physical information that can be represented in numerical values such as acceleration as illustrated in FIG. 3 (3-1), heart rate, body temperature, blood pressure, blood oxygen saturation (SpO₂), respiration rate, acceleration (active mass), the number of steps, and the number of going up and down as illustrated in FIG. 3 (3-2), and image information such as video images in which behavior of the patient P is captured.

Then, the text data generation device 20 estimates time-series states of the patient P from the acquired measurement data of the patient (step S2). For example, as time-series states of the patient P, the text data generation device 20 estimates restlessness, awakeness, sleep, active mass, irregular pulse, cardiac function, respiratory function, cerebral function, stress, GCS, and JCS as illustrated in FIG. 4.

Then, the text data generation device 20 generates text data representing the state of the patient P in a given time period, from the estimated time-series states of the patient P (step S3). For example, as illustrated in FIG. 5 (5-1), the text data generation device 20 generates text data representing the state of the patient P for each hour, or generates text data representing the states of the patient Pin a day in a sentence as illustrated in FIGS. 5 (5-2) and (5-3).

Thereafter, the text data generation device 20 displays and outputs the generated text data representing the state of the patient P for each time period on the display device 12 so as to allow the nurse taking care of the patient P to view it (step S4). At that time, the text data generation device 20 outputs the text data displayed in the text box to be revisable, as illustrated in FIGS. 6 and 7. Note that the text data generation device 20 displays measurement data of the patient P together with the text data. The measurement data displayed together with the text data is not limited to physical information such as acceleration as illustrated in FIG. 6, but may be image information in which the patient P is captured.

Then, when the text data displayed in the text box is revised by the nurse, the text data generation device 20 accepts the input of the revised text data (step S4). Then, when the nurse performs a registration operation of the text data, the text data generation device 20 registers the text data input in the text box with the electronic medical record of the patient P (step S5). As described above, in the present embodiment, the text data generation device 20 estimates the time-series states of the patient P from the time-series measurement data measured from the patient P, and generates text data representing the state of the patient P in a predetermined time period from the estimated states of the patient P. Since the text data generation device 20 automatically generates text data of the state of the patient P in a predetermined time period in this manner, it is unnecessary to describe the state of the patient P in text by a nurse or the like. This enables a recording operation on a medical record or the like to be simplified. Therefore, it is possible to mitigate a workload of recording the state of the patient P on a medical record or the like by the nurses and the like, and suppress erroneous record, and also possible to provide appropriate nursing later while referring to the record of the state of the patient P by the nurses and the like.

### Second Exemplary Embodiment

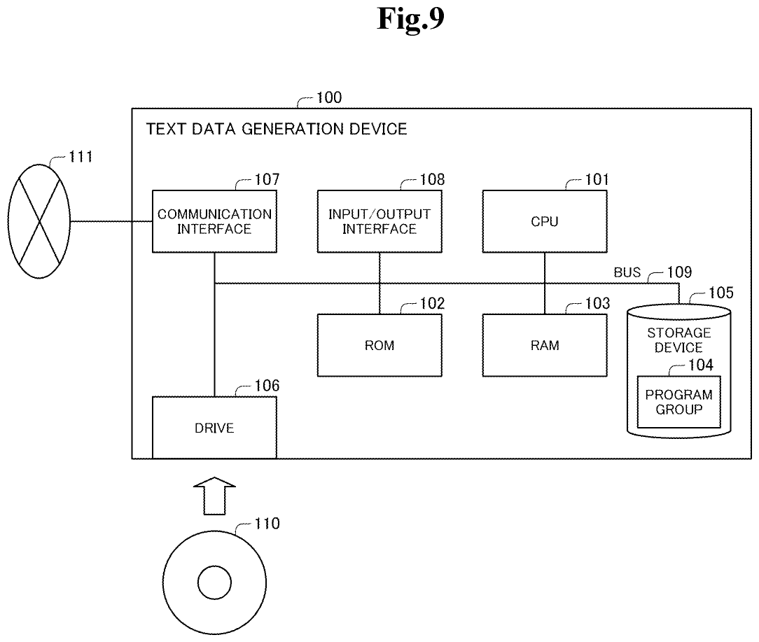

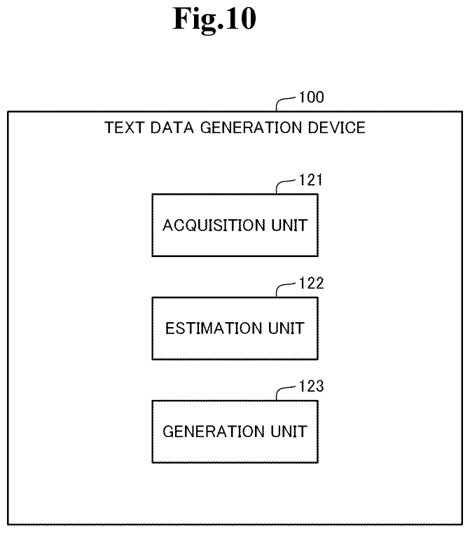

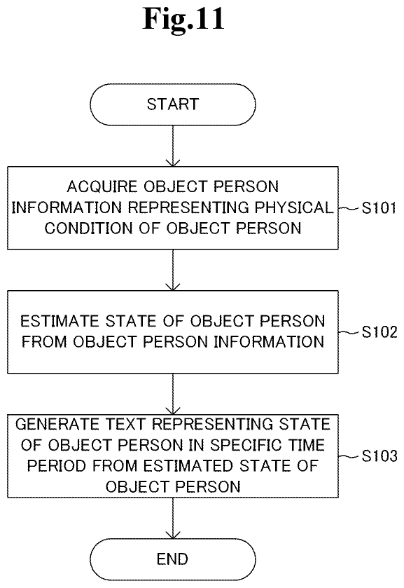

Next, a second exemplary embodiment of the present invention will be described with reference to FIGS. 9 to 11. FIGS. 9 and 10 are block diagrams illustrating a configuration of a text data generation device according to the second exemplary embodiment, and FIG. 11 is a flowchart illustrating an operation of the text data generation device. Note that the present embodiment shows the outlines of the configurations of the text data generation device and the text data generation method described in the embodiment described above.

First, a hardware configuration of the text data generation device 100 in the present embodiment will be described with reference to FIG. 9. The text data generation device 100 is configured of a typical information processing device, having a hardware configuration as described below as an example.
Central Processing Unit (CPU) 101 (arithmetic device)
Read Only Memory (ROM) 102 (storage device)
Random Access Memory (RAM) 103 (storage device)
Program group 104 to be loaded to the RAM 103
Storage device 105 storing therein the program group 104
Drive 106 that performs reading and writing on a storage medium 110 outside the information processing device
Communication interface 107 connecting to a communication network 111 outside the information processing device
Input/output interface 108 for performing input/output of data
Bus 109 connecting the respective constituent elements

The text data generation device 100 can construct, and can be equipped with, an acquisition unit 121, an estimation unit 122, and a generation unit 123 illustrated in FIG. 10 through acquisition and execution of the program group 104 by the CPU 101. Note that the program group 104 is stored in the storage device 105 or the ROM 102 in advance, and is loaded to the RAM 103 and executed by the CPU 101 as needed. Further, the program group 104 may be provided to the CPU 101 via the communication network 111, or may be stored on the storage medium 110 in advance and read out by the drive 106 and supplied to the CPU 101. However, the acquisition unit 121, the estimation unit 122, and the generation unit 123 may be constructed by dedicated electronic circuits for implementing such means.

Note that FIG. 9 illustrates an example of a hardware configuration of an information processing device that is the text data generation device 100. The hardware configuration of the information processing device is not limited to that described above. For example, the information processing device may be configured of part of the configuration described above, such as without the drive 106.

The text data generation device 100 executes the text data generation method illustrated in the flowchart of FIG. 11, by the functions of the acquisition unit 121, the estimation unit 122, and the generation unit 123 constructed by the program as described above.

As illustrated in FIG. 11, the text data generation device 100 executes processing to:
acquire object person information representing time-series physical condition of an object person (step S101),
estimate time-series states of the object person on the basis of the object person information (step S102), and
on the basis of the estimated time-series states of the object person, generate text data representing the state of the object person in a given time period (step S103).

With the configuration described above, in the present invention, the text data generation device 100 acquires time-series object person information measured from an object person who is a patient, estimates time-series states of the object person, and generates text data representing the state of the object person in a predetermined time period from the estimated states. As described above, since the text data generation device 100 automatically generates text data of the state of the object person in a predetermined time period, in the case where the object person is a patient for example, it is unnecessary to describe the state of the object person in text by a nurse. This enables an operation of recording on a medical record or the like to be simplified. Therefore, it is possible to mitigate a workload of recording the state of an object person on a medical record or the like by a nurse or the like, and suppress erroneous record, and also possible to provide appropriate nursing while referring to the record.

Note that the program described above can be supplied to a computer by being stored on a non-transitory computer-readable medium of any type. Non-transitory computer-readable media include tangible storage media of various types. Examples of non-transitory computer-readable media include magnetic storage media (for example, flexible disk, magnetic tape, and hard disk drive), magneto-optical storage media (for example, magneto-optical disk), a CD-ROM (Read Only Memory), a CD-R, a CD-R/W, and semiconductor memories (for example, mask ROM, PROM (Programmable ROM), EPROM (Erasable PROM), flash ROM, and RAM (Random Access Memory)). The program may be supplied to a computer by a transitory computer-readable medium of any type. Examples of transitory computer-readable media include electric signals, optical signals, and electromagnetic waves. A transitory computer-readable medium can supply a program to a computer via a wired communication channel such as a wire and an optical fiber, or a wireless communication channel.

While the present invention has been described with reference to the exemplary embodiments described above, the present invention is not limited to the above-described embodiments. The form and details of the present invention can be changed within the scope of the present invention in various manners that can be understood by those skilled in the art. Moreover, at least one of the functions of the acquisition unit 121, the estimation unit 122, and the generation unit 123 described above may be carried out by an information processing device provided and connected to any location on the network, that is, may be carried out by so-called cloud computing.

### <Supplementary Notes>

The whole or part of the exemplary embodiments disclosed above can be described as the following supplementary notes. Hereinafter, outlines of the configurations of a text data generation method, a text data generation device, and a program, according to the present invention, will be described. However, the present invention is not limited to the configurations described below.

### (Supplementary Note 1)

A text data generation method comprising:
acquiring object person information representing time-series physical condition of an object person;
estimating time-series states of the object person on a basis of the object person information; and
generating text data representing a state of the object person in a given time period on a basis of the estimated time-series states of the object person.

### (Supplementary Note 2)

The text data generation method according to supplementary note 1, further comprising
generating text data representing a state of the object person in each of a plurality of the given time periods.

### (Supplementary Note 3)

The text data generation method according to supplementary note 2, further comprising
on a basis of the state of the object person in each of the plurality of the given time periods, generate text data representing a state of the object person in a time period in which the plurality of the given time periods are combined.

### (Supplementary Note 4)

The text data generation method according to any of supplementary notes 1 to 3, further comprising
generating text data representing the state of the object person in the given time period on a basis of the estimated state of the object person at a predetermined time with respect to the given time period.

### (Supplementary Note 5)

The text data generation method according to any of supplementary notes 1 to 4, further comprising
registering the text data representing the state of the object person in the given time period with a data file set to the object person, together with time data specifying the given time period.

### (Supplementary Note 6)

The text data generation method according to supplementary note 5, further comprising
displaying and outputting the text data so as to be viewable by a registered person having a predetermined relation with the object person, and then, when a registration operation is performed by the registered person, registering the text data with the data file.

### (Supplementary Note 7)

The text data generation method according to supplementary note 6, further comprising
displaying and outputting the text data to be revisable, and then, when the text data is revised and a registration operation is performed by the registered person, registering the revised text data with the data file.

### (Supplementary Note 8)

The text data generation method according to supplementary note 6 or 7, further comprising
when displaying and outputting the text data, displaying and outputting the object person information acquired from the object person together with the text data.

### (Supplementary Note 9)

The text data generation method according to supplementary note 8, further comprising
displaying and outputting the text data generated for each of a plurality of the given time periods in a time-series manner, and displaying and outputting the object person information acquired from the object person in a time-series manner, together with the text data.

### (Supplementary Note 10)

The text data generation method according to any of supplementary notes 1 to 9, further comprising
registering the object person information with a data file set to the object person, together with information corresponding to a time at which the object person information is acquired.

### (Supplementary Note 11)

A text data generation device comprising:
an acquisition unit that acquires object person information representing time-series physical condition of an object person;
an estimation unit that estimates time-series states of the object person on a basis of the object person information; and
a generation unit that generates text data representing a state of the object person in a given time period on a basis of the estimated time-series states of the object person.

### (Supplementary Note 12)

The text data generation device according to supplementary note 11, wherein
the generation unit generates text data representing a state of the object person in each of a plurality of the given time periods.

### (Supplementary Note 13)

The text data generation device according to supplementary note 12, wherein
on a basis of the state of the object person in each of the plurality of the given time periods, the generation unit generates text data representing a state of the object person in a time period in which the plurality of the given time periods are combined.

### (Supplementary Note 14)

The text data generation device according to any of supplementary notes 11 to 13, wherein
the generation unit generates text data representing the state of the object person in the given time period on a basis of the estimated state of the object person at a predetermined time with respect to the given time period.

### (Supplementary Note 15)

The text data generation device according to any of supplementary notes 11 to 14, further comprising
a registration unit that registers the text data representing the state of the object person in the given time period with a data file set to the object person, together with time data specifying the given time period.

### (Supplementary Note 16)

The text data generation device according to supplementary note 15, wherein
the registration unit displays and outputs the text data so as to be viewable by a registered person having a predetermined relation with the object person, and then, when a registration operation is performed by the registered person, the registration unit registers the text data with the data file.

### (Supplementary Note 17)

The text data generation device according to supplementary note 16, wherein
the registration unit displays and outputs the text data to be revisable, and then, when the text data is revised and a registration operation is performed by the registered person, the registration unit registers the revised text data with the data file.

### (Supplementary Note 18)

The text data generation device according to supplementary note 16 or 17, wherein
when displaying and outputting the text data, the registration unit displays and outputs the object person information acquired from the object person together with the text data.

### (Supplementary Note 19)

The text data generation device according to supplementary note 18, wherein
the registration unit displays and outputs the text data generated for each of a plurality of the given time periods in a time-series manner, and displays and outputs the object person information acquired from the object person in a time-series manner, together with the text data.

### (Supplementary Note 20)

The text data generation device according to any of supplementary notes 11 to 19, wherein
the registration unit registers the object person information with a data file set to the object person, together with information corresponding to a time at which the object person information is acquired.

### (Supplementary Note 21)

A computer-readable medium storing thereon a program for causing an information processing device to execute processing to:

acquire object person information representing time-series physical condition of an object person;

estimate time-series states of the object person on a basis of the object person information; and

generate text data representing a state of the object person in a given time period on a basis of the estimated time-series states of the object person.

### REFERENCE SIGNS LIST

1 sensor
2 communication device
10 text data generation system
11 input device
12 display device
20 text data generation device
21 acquisition unit
22 estimation unit
23 generation unit
24 registration unit
26 measurement data storage unit
27 state data storage unit
28 text data storage unit
29 patient data storage unit
100 text data generation device
101 CPU
102 ROM
103 RAM
104 program group
105 storage device
106 drive
107 communication interface
108 input/output interface
109 bus
110 storage medium
111 communication network
121 acquisition unit
122 estimation unit
123 generation unit

What is claimed is:

1. A text data generation method comprising:
acquiring object person information representing time-series physical condition of an object person, the object person information including image data of the object person;
estimating time-series states of the object person on a basis of the object person information; and
generating text data representing a state of the object person in a given time period on a basis of the estimated time-series states of the object person.

2. The text data generation method according to claim 1, further comprising
generating text data representing a state of the object person in each of a plurality of the given time periods.

3. The text data generation method according to claim 2, further comprising
on a basis of the state of the object person in each of the plurality of the given time periods, generate text data representing a state of the object person in a time period in which the plurality of the given time periods are combined.

4. The text data generation method according to claim 1, further comprising
generating text data representing the state of the object person in the given time period on a basis of the estimated state of the object person at a predetermined time with respect to the given time period.

5. The text data generation method according to claim 1, further comprising
registering the text data representing the state of the object person in the given time period with a data file set to the object person, together with time data specifying the given time period.

6. The text data generation method according to claim 5, further comprising
displaying and outputting the text data so as to be viewable by a registered person having a predetermined relation with the object person, and then, when a registration operation is performed by the registered person, registering the text data with the data file.

7. The text data generation method according to claim 6, further comprising
displaying and outputting the text data to be revisable, and then, when the text data is revised and a registration operation is performed by the registered person, registering the revised text data with the data file.

8. The text data generation method according to claim 6, further comprising
when displaying and outputting the text data, displaying and outputting the object person information acquired from the object person together with the text data.

9. The text data generation method according to claim 8, further comprising
displaying and outputting the text data generated for each of a plurality of the given time periods in a time-series manner, and displaying and outputting the object person information acquired from the object person in a time-series manner, together with the text data.

10. The text data generation method according to claim 1, further comprising
registering the object person information with a data file set to the object person, together with information corresponding to a time at which the object person information is acquired.

11. A text data generation device comprising:
at least one memory configured to store instructions; and
at least one processor configured to execute instructions to:
acquire object person information representing time-series physical condition of an object person;
estimate time-series states of the object person on a basis of the object person information; and
generate text data representing a state of the object person in a given time period on a basis of the estimated time-series states of the object person.

12-20. (canceled)

21. A non-transitory computer-readable medium storing thereon a program comprising instructions for causing an information processing device to execute processing to:
acquire object person information representing time-series physical condition of an object person;
estimate time-series states of the object person on a basis of the object person information; and
generate text data representing a state of the object person in a given time period on a basis of the estimated time-series states of the object person.

22. The text data generation method according to claim 1, further comprising
generating the text data by using the object person information and the estimated state of the object person, and a learning model generated by machine learning to learn a relationship between object person information and a state that were recorded for the object person in past and text data.

23. The text data generation method according to claim 7, further comprising
displaying and outputting the text data for decision making by medical professionals on whether or not revision of the text data is necessary.
